(19) United States
(12) Patent Application Publication
(10) Pub. No.: US 2007/0017519 A1
(43) Pub. Date: Jan. 25, 2007

(54) PROCESS AND DEVICE FOR SETTING A GAS FLOW DISTRIBUTION OF A BREATHING GAS MIXTURE IN A BRANCHING BREATHING GAS LINE

(75) Inventor: Artur CHUNG-CHE KUO, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: DRAGERWERK AKTIENGESELLSCHAFT, Lubeck (DE)

(21) Appl. No.: 11/420,364

(22) Filed: May 25, 2006

(30) Foreign Application Priority Data
Jul. 23, 2005 (DE) 10 2005 034 538.7

(51) Int. Cl. A62B 7/04; A61M 16/00

(52) U.S. Cl. 128/204.26

(57) ABSTRACT

An improved process and a device are provided, with which it is possible in a targeted manner to achieve deflection of a gas flow distribution flowing in linearly into the branch, especially in the area of the trachea and the lung of a patient, without mechanical components at the site of the branch.

### CROSS REFERENCE TO RELATED APPLICATIONS

This application claims the benefit of priority under 35 U.S.C. § 119 of German Patent Application DE 10 2005 034 538.7 filed Jul. 23, 2005, the entire contents of which are incorporated herein by reference.

### FIELD OF THE INVENTION

The present invention pertains to a process for setting a gas flow distribution of a breathing gas mixture in a branching breathing gas line according to claim 1 as well as to a device for carrying out the process.

### BACKGROUND OF THE INVENTION

To set the direction of flow in a pipeline having a branch, it has hitherto been necessary to use mechanical or electromechanical means such as valves, adjusting elements or guide blades with associated control means. It is practically impossible to accommodate such components at or in the patient in case of the respiration and supply of patients with gases.

To supply a lung segment, attempts have hitherto been made either to pump in a large volume flow under high pressure or to reach the corresponding bronchial branch with a long tube. Both are associated with risks.

### SUMMARY OF THE INVENTION

Thus, the object of the present invention is to make available an improved process and a device by means of which it is possible in a targeted manner to achieve deflection of a gas flow distribution flowing linearly into a branch, especially in the area of the human trachea and lung, without mechanical components at the site of the branch.

According to the invention, a process is provided for setting a gas flow distribution of a breathing gas mixture in a branching breathing gas line by setting the velocity profile and/or the viscosity profile of the breathing gas flow over the cross section of the breathing gas line such that the breathing gas flow is deflected in the direction of the branch following in the direction of flow with increasing velocity and/or increasing viscosity.

The velocity and/or viscosity profile may be generated with gas flows led in parallel in the breathing gas line. The gas flows may contain air, nitrogen, carbon dioxide, laughing gas (N₂O), helium and/or one or more noble gases.

The breathing gas mixture may contain a gaseous, aerosol type or particulate medication, anesthetic or a labeling substance, which are dispensed especially into the parts of the breathing gas mixture that are deflected into the branch.

The branching breathing gas line may be formed from a breathing tube in the human trachea with a first or next branch into the lung.

The velocity of the breathing gas mixture may advantageously be 0.3 to 60 m per second.

The composition and/or the velocity of the breathing gas flow may be changed over the cross section of the breathing gas line in a time-dependent manner, especially periodically.

According to another aspect of the invention, a device is provided for setting a gas flow distribution of a breathing gas mixture in a branching breathing gas line by setting the velocity and/or viscosity profile of the breathing gas flow over the cross section. The device comprises a gas dispensing means with a plurality of parallel gas lines in individual channels in the breathing gas line. Gas sources are provided for the gas dispensing means. A measuring and control unit is provided for setting the breathing gas flow. A display or detection unit is also provided.

The device may have parallel gas lines designed in the form of a multilumen breathing tube, especially in the form of a two- to four-lumen breathing tube.

An essential advantage of the process and of the device according to the invention is that it is possible to perform a targeted deflection of a gas flow into a branch or even into a plurality of consecutive branches of the lung, for example, from the trachea specifically into a lobe of the lung or into an area of the lung to which, for example, a breathing gas mixture rich in oxygen, a medication or a tracing substance is to be admitted for therapeutic or diagnostic purposes, without mechanical or electromechanical components in the patient.

Exemplary embodiments of the present invention will be explained below on the basis of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1A is a cross sectional view of a multilumen breathing tube;

FIG. 1B is a cross sectional view of a multilumen breathing tube;

FIG. 1C is a cross sectional view of a multilumen breathing tube;

FIG. 2A is an alternative embodiment of the cross section from FIG. 1C;

FIG. 2B is an alternative embodiment of the cross section from FIG. 1C;

FIG. 3 is a schematic view of a device according to the invention for carrying out the process according to the invention;

FIG. 4A is a breathing gas line with branches each branching off by 30° on both sides in the lung with a breathing tube in the human windpipe (trachea);

FIG. 4B is a cross sectional view of the breathing tube of FIG. 4A; and

FIG. 5 is a view showing the isolines of the gas flow velocity for the breathing gas line shown.

### DESCRIPTION OF THE PREFERRED EMBODIMENTS

Referring to the drawings in particular, this process with the associated device is especially suitable for specifically directing a direction-controlled breathing gas flow into a diseased lung area that is to be treated or labeled via one branch or consecutive branches. For example, a collapsed lung area can be pumped up and respirated again, while a complementary lung area is spared. As an alternative, a medication or a labeling substance can be applied in a targeted manner.

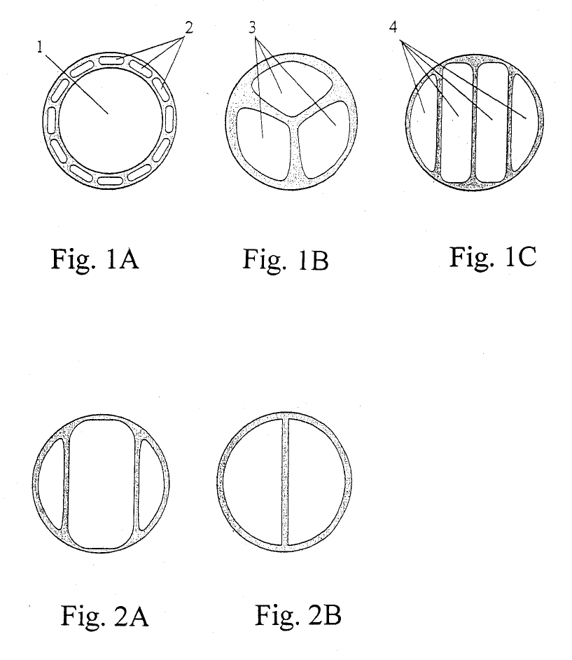

An asymmetrical, i.e., inhomogeneous distribution of the gas concentration and hence of the viscosity and/or the gas velocity of a breathing gas flow can be brought about by means of a multilumen breathing tube or catheter. A multilumen breathing tube has the standardized external diameter of a tube and a plurality of parallel channels, which are arranged within the tube. The partial flows of the breathing gas flow are set individually. Examples of possible cross sections are shown in FIG. 1A-1B with channels 1, 2, 3, 4 for guiding and setting the velocity and/or viscosity profiles. In FIG. 1A, about 2 to 12 side channels 2 are integrated in the tube wall and distributed homogeneously around the main channel 1. The cross section of a special three-lumen tube is shown in FIG. 1B and FIG. 1C schematically shows a breathing tube with four channels. The individual channels can be combined, so that other three-lumen catheters can be formed from the four channel breathing tube FIG. 1C and FIGS. 2A and 2B. To keep the flow resistance as low as possible, the cross sections of all channels should be as large as possible and the partitions as thin as possible.

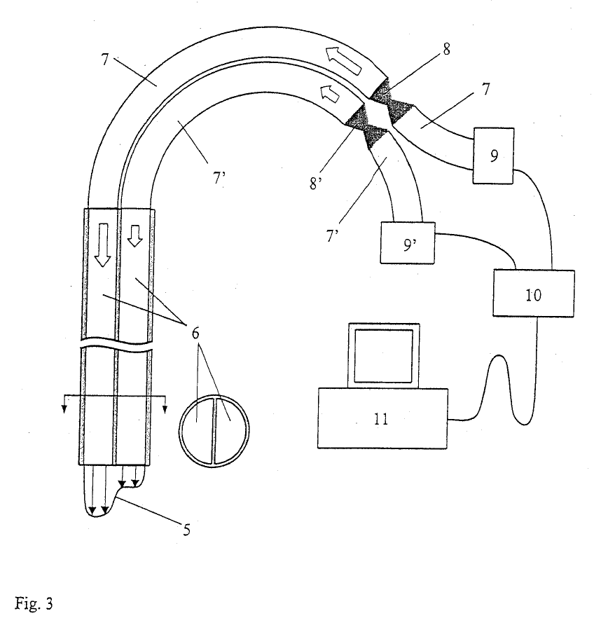

FIG. 3 schematically shows a device for carrying out the process, with which the gas flow velocity or viscosity of the gas flow in the individual channels can be set by means of a two-lumen breathing tube corresponding to the cross section of FIG. 2B such that a velocity and/or viscosity profile 5 is obtained. The individual channels 6 in the tube are supplied via flexible tubes 7, 7′ with valves or gas-dispensing means 8, 8′ and with corresponding separate gas sources 9, 9′. The admixing of gas and the optionally measured volume flows are set by means of a measuring and control unit 10. The measuring and control unit 10 is optionally connected to a display or detection unit 11, for example, a PC/monitor or even an anesthesia apparatus or respirator. Medications, anesthetics or a labeling substance are advantageously dispensed via the gas dispensing means 8, 8′ into the individual channel 6 with the higher gas flow velocity or gas viscosity such that as a consequence of the subsequent branching into or in the lung, the active ingredients will specifically enter the desired area to be treated or labeled, e.g., for an imaging diagnostic procedure.

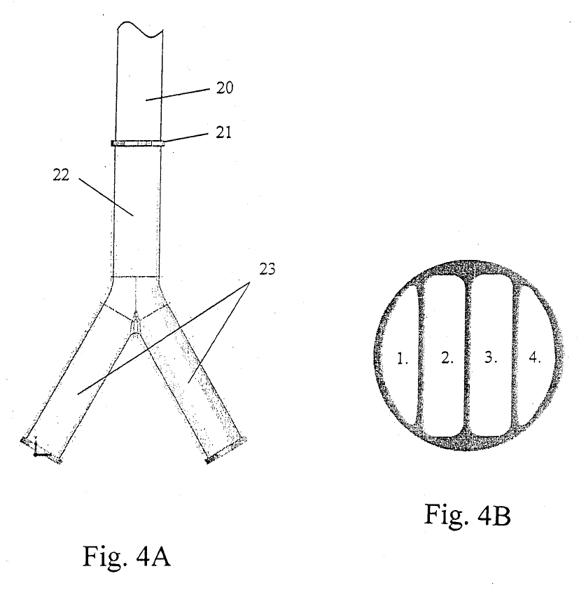

FIGS. 4A and 4B show the functionality of the process by means of a measuring instrument. A tube 20 with the end 21 and with the four-lumen cross section shown in FIG. 4B forms, together with section 22 of a trachea, the linear inlet section for the breathing gas flow. The adjoining branch to the left and right with 30° each in respect to the inlet section is the feed line to the lung lobes via the bronchial branches 23. The tube 20 has a length of especially 350 mm and a diameter of 18 mm. The diameter and the length of section 22 of the trachea are 18 mm and 55 mm. The diameter and the length of the two bronchial branches 23 are 15 mm and 70 mm. The transition from the trachea to the bronchial branches 23 is continuous. The data of the model correspond to those of the human lung at the first branch in the lung. The branches are arranged symmetrically to the left and right and form an angle of 60°.

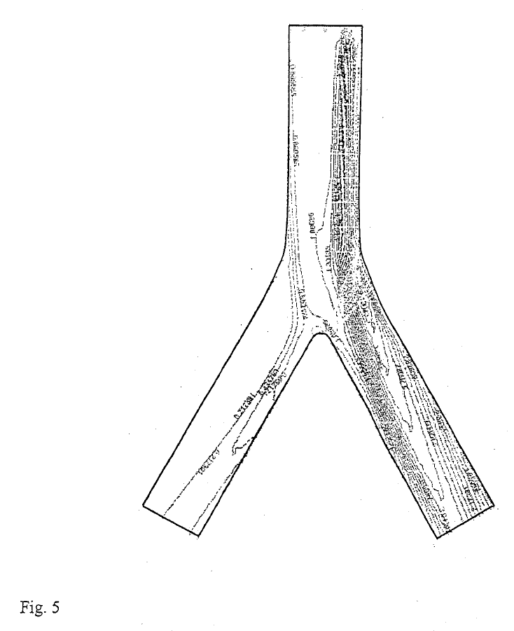

The following measured results were obtained with the measuring instrument shown in FIG. 4. An air flow is first introduced stationarily into all four channels of the tube 20. The constant velocities in the four channels are, following the numbering of the channels in FIG. 4B from left to right (1, 2, 3, 4), 1, 1, 1 as well as 5 m per second. The result in FIG. 5 (isolines of the gas flow velocity) shows that the main flow was strongly deflected to the right because of the Coanda effect. There is a flow of 21.3 L per minute through the right branch and 2.2 L per minute through the left branch, i.e., the flow on the right-hand side is nearly 10 times that on the left-hand side.

As an alternative, measurements were performed with an instationary gas flow. Flow was admitted into the channels 1, 2, 3 in FIG. 4B at a constant velocity of 1 m per sec. The velocity of the gas in channel 4 changed every 0.2 sec between 1 and 5 m per second corresponding to a change in the total volume flow from 13 to 23.5 L per minute. The amount of air and the breathing gas volume that enters the right branch over one period was four times that entering the left branch.

As an alternative, the deflection due to the admixing of another gas, here helium, was investigated. Helium was sent here through channel 4 in FIG. 4 at a constant velocity of 1 m per second, while air was flowing through the other channels 1, 2, 3 at a constant velocity of 1 m per second. The main flow is distributed farther to the right because of the difference in viscosity. It was measured that the volume flow percentage of helium through the right branch, equaling 0.39 L per minute, is nearly 40 times that flowing through the left branch, which equals 0.01 L per minute. The shown distribution of the volume and mass flow percentages confirms the possibility of transporting therapeutically or diagnostically effective substances specifically locally into the lung by means of a carrier gas such as helium.

In another alternative experiment, air was admitted into the channels 1, 2, 4 in FIG. 4B and helium was admitted into channel 3, and the gas flow velocity was 1 m per second in all four channels. Because of the vortex structures rolling up in the shear layer and the interaction of these structures with the boundary layer, the main flow was slightly deflected to the left in this case, so that the volume flow of the gas mixture is about 50% higher on the left with 8.9 L per minute than in the right branch with 6 L per minute, but it is nevertheless seen that the volume flow percentage of helium is still twice as high on the right-hand side with 0.79 L per minute than on the left-hand side with 0.38 L per minute.

While specific embodiments of the invention have been shown and described in detail to illustrate the application of the principles of the invention, it will be understood that the invention may be embodied otherwise without departing from such principles.

What is claimed is:

1. A process for setting a gas flow distribution of a breathing gas mixture in a branching breathing gas line, the process comprising:
setting the velocity profile and/or the viscosity profile of a breathing gas flow over a cross section of the breathing gas line such that the breathing gas flow is deflected in the direction of a branch following in the direction of flow with increasing velocity and/or increasing viscosity.

2. A process in accordance with claim 1, wherein the velocity and/or viscosity profile is generated with gas flows led in parallel in the breathing gas line.

3. A process in accordance with claim 2, wherein the gas flow contains one or more of air, nitrogen, carbon dioxide, laughing gas (N₂O), helium and/or one or more noble gases.

4. A process in accordance with claim 1, wherein the breathing gas mixture contains a gaseous, aerosol type or particulate medication, anesthetic or a labeling substance, which is dispensed into the parts of the breathing gas mixture that are deflected into the branch.

5. A process in accordance with claim 1, wherein the branching breathing gas line is formed from a breathing tube placed in the human trachea with a first or next branch into the lung.

6. A process in accordance with claim 1, wherein the velocity of the breathing gas mixture is 0.3 to 60 m per second.

7. A process in accordance with claim 1, wherein the composition and/or the velocity of the breathing gas flow is changed over the cross section of the breathing gas line in a time-dependent manner.

8. A process in accordance with claim 1, wherein the composition and/or the velocity of the breathing gas flow is periodically changed over the cross section of the breathing gas line.

9. A device for setting a gas flow distribution of a breathing gas mixture, the device comprising:
a branching breathing gas line including a main line and a first branch and a second branch;
a gas dispensing means with a plurality of parallel gas lines provided as individual channels in the breathing gas line;
gas sources for the gas dispensing means;
a measuring and control unit for setting the breathing gas flow wherein the gas flow distribution of a breathing gas mixture is set by setting the velocity and/or viscosity profile of the breathing gas flow over the cross section.

10. A device in accordance with claim 9, further comprising: a display or detection unit.

11. A device in accordance with claim 9, wherein the parallel gas lines are designed in the form of a multilumen breathing tube.

12. A device in accordance with claim 9, wherein two- to four-lumen are provided in the breathing tube.

13. A process comprising:
providing a branching breathing gas line including a main line branching to a first branch and a second branch;
providing a plurality of parallel gas lines as individual channels in the breathing gas line;
providing gas sources and feeding gas into the parallel gas lines;
setting the breathing gas flow of the gas fed into the parallel gas lines so a gas flow distribution of a breathing gas mixture is set by setting the velocity and/or viscosity profile of the breathing gas flow over the cross section of the breathing gas line.

14. A process in accordance with claim 13, wherein the velocity and/or viscosity profile is generated with gas flows led in parallel in the breathing gas line.

15. A process in accordance with claim 14, wherein the gas flow contains one or more of air, nitrogen, carbon dioxide, laughing gas (N₂O), helium and/or one or more noble gases.

16. A process in accordance with claim 13, wherein the breathing gas mixture contains a gaseous, aerosol type or particulate medication, anesthetic or a labeling substance, which is dispensed into the parts of the breathing gas mixture that are deflected into the branch.

17. A process in accordance with claim 13, wherein the branching breathing gas line is formed from a breathing tube placed in the human trachea with a first branch placed into the lung.

18. A process in accordance with claim 13, wherein the velocity of the breathing gas mixture is 0.3 to 60 m per second.

19. A process in accordance with claim 13, wherein the composition and/or the velocity of the breathing gas flow is changed over the cross section of the breathing gas line in a time-dependent manner.

20. A process in accordance with claim 13, wherein the composition and/or the velocity of the breathing gas flow is periodically changed over the cross section of the breathing gas line.
